(19) United States
(12) Patent Application Publication
(10) Pub. No.: US 2009/0123576 A1
(43) Pub. Date: May 14, 2009

(54) ACTIVE SUBSTANCE COMBINATIONS OF ANISE FRUIT EXTRACT AND WHITE TEA EXTRACT

(75) Inventors: Rainer Kroepke, Schenefeld (DE); Sabine Faenger, Hamburg (DE); Christopher Mummert, Bienenbuettel (DE); Jens Schulz, Rellingen (DE)

Correspondence Address: GREENBLUM & BERNSTEIN, P.L.C., 1950 ROLAND CLARKE PLACE, RESTON, VA, 20191, US

(73) Assignee: Beiersdorf AG, Hamburg (DE)

(21) Appl. No.: 12/258,934

(22) Filed: Oct. 27, 2008

(30) Foreign Application Priority Data
Nov. 1, 2007 (DE) 10 2007 052 536.4

(51) Int. Cl. A61K 8/97; A61K 8/06; A61Q 19/00; A61Q 19/06; A61Q 19/08

(52) U.S. Cl. 424/729; 424/777

(57) ABSTRACT

An active substance combination which comprises anise fruit extract and white tea extract and a cosmetic preparation comprising same. This Abstract is not intended to define the invention disclosed in the specification, nor intended to limit the scope of the invention in any way.

### CROSS-REFERENCE TO RELATED APPLICATIONS

The present application claims priority under 35 U.S.C. § 119 of German Patent Application No. 10 2007 052 536.4, filed Nov. 1, 2007, the entire disclosure of which is expressly incorporated by reference herein.

### BACKGROUND OF THE INVENTION

1. Field of the Invention

The present invention relates to active substance combinations of an effective amount of anise fruit extract and white tea extract and cosmetic preparations containing such active substance combinations.

2. Discussion of Background Information

Cosmetics can cover all measures that make changes to skin and hair for esthetic reasons or are used for cleansing the skin. Cosmetics therefore mean caring for, improving and beautifying the outward appearance of the body in order to please others as well as oneself in a manner that can be seen, felt and smelled.

The aim of skin care is furthermore to compensate for the fat and water lost by the skin due to daily washing. This is important precisely when the natural regeneration capacity is insufficient. In addition, skin care products are to protect against environmental influences, in particular against sun and wind and to delay skin aging.

Chronological skin aging is caused by e.g., endogenous, genetically determined factors. Age-related structural damage and dysfunctions occur in the epidermis and dermis of the skin, which can also be referred to as “senile xerosis:”.

a) Dryness, roughness and development of dry lines/wrinkles

### b) Itching

c) Reduced rehydration by sebaceous glands (e.g. after washing).

Exogenous factors such as UV light and chemical noxa can act cumulatively and, e.g., accelerate or aggravate endogenous aging processes. In particular, exogenous influences can cause the following structural damage and dysfunctions in the epidermis and dermis of the skin, which go beyond the level and quality of the damage in chronological aging:

d) Visible vascular dilatations (telangiectasis, cuperosis),
e) Slackness and formation of wrinkles,
f) Local hyper-, hypo- and mispigmentations (e.g., age marks) and
g) Increased susceptibility to mechanical stress (e.g., tendency to crack).

The present invention relates in particular to products for the care of skin aged in a natural manner as well as to the phenomena listed under a), e) and g).

Products for the care of slack, in particular aged skin, are known per se. They contain, e.g., retinoids (vitamin A acid and/or its derivatives) or vitamin A and/or its derivatives. The effect of these products on structural damage is limited in scope, however. Moreover, there are considerable difficulties during product development in stabilizing the active ingredients sufficiently against oxidative decomposition. In addition, vitamin A acid-containing products can cause pronounced erythematous skin irritations. Retinoids can therefore be used only in low concentrations.

Flaccid skin is often also associated with an accompanying phenomenon of excess weight and/or so-called cellulite frequently associated therewith.

The body-consciousness of consumers has increased significantly in recent years. In this connection, in addition to cleansing and care applications, measures are also increasingly being taken to improve the silhouette of the body. Cellulite—a widespread phenomenon—is thereby of central importance. The visible appearance of cellulite is based on an increase of fatty bodies in the subcutis (subcutaneous fatty tissue), a weakness of the connective tissue and a reduction of the perfusion ratios in the blood stream and lymphatic tract. The cause is thus a partially constitutional weakening of the connective tissue with the simultaneous occurrence of enlarged fat cell chambers as a result of excess weight, an unbalanced diet, lack of exercise. The formation of cellulite can furthermore be attributed to increased permeability of the capillary walls which permits the penetration of water into the connective tissue.

In addition, there can be a local testosterone deficiency at the affected areas of the skin. In any case, cellulite is a phenomenon that is virtually never observed in men.

In view of the foregoing, it would be desirable to find ways to avoid the disadvantages of the prior art. In particular it would be desirable to have available preparations that can effect an advantageous tightening of flaccid skin.

It would also be desirable to provide skin care preparations and preparations for care of the skin which care for and beautify the outward appearance of the body. These preparations should in particular also improve the moisture of the skin.

Surprisingly, it has been found that the use of a cosmetic preparation which comprises a cosmetically effective amount of anise fruit extract and white tea extract remedies the disadvantages of the prior art.

### SUMMARY OF THE INVENTION

The present invention provides an active substance combination which comprises anise fruit extract and white tea extract and a cosmetic preparation which comprises an effective amount of the active substance combination.

In one aspect, the preparation may comprise from about 0.0001% to about 30% by weight, e.g., from about 0.5% to about 10% by weight of anise fruit extract, based on the total weight of the preparation.

In another aspect, the preparation may comprise from about 0.0001% to about 30% by weight, e.g., from about 0.5% to about 10% by weight of white tea extract, based on the total weight of the preparation.

In yet another aspect of the preparation or the active substance combination, the weight ratio anise fruit extract:white tea extract may be from about 1:10 to about 10:1 e.g., from about 1:5 to about 5:1, or from about 1:2 to about 2:1.

In a still further aspect, the preparation or active substance combination may comprise anise fruit extract and one or more substances selected from gallocatechol, epigallocatechol, epicatechol, epigallocatechol gallate, gallocatechol gallate, epicatechol gallate, catechol, quercetin, and a methyl xanthine (such as, e.g., one or more of caffeine, theophylline and theobromine).

In yet another aspect, the preparation or active substance combination may comprise an aqueous anise fruit extract and/or may be present in the form of an emulsion.

The present invention also provides a cosmetic preparation which comprises from about 0.1% to about 30% by weight of an aqueous anise fruit extract and from about 0.1% to about 30% by weight of white tea extract, based on the total weight of the preparation, the weight ratio anise fruit extract:weight tea extract being from about 5:1 to about 1:5.

In one aspect, the preparation may comprise from about 0.5% to about 20% by weight of an aqueous anise fruit extract and from about 0.5% to about 20% by weight of white tea extract. In another aspect, the preparation may be present as an O/W emulsion.

The present invention also provides a method of increasing the moisture content of skin or moisturizing skin. The method comprises applying to at least parts of the skin the preparation of the present invention as set forth above (including the various aspects thereof) in an amount which is effective to increase the moisture content of the skin and/or to moisturize the skin.

The present invention also provides a method of improving the surface structure of skin. The method comprises applying to at least parts of the skin the preparation of the present invention as set forth above (including the various aspects thereof) in an amount which is effective for improving the surface structure of the skin (in particular, for smoothing the skin and/or for reducing indentations in the skin).

The present invention also provides a method of reducing cellulite. The method comprises applying to at least parts of the skin the preparation of the present invention as set forth above (including the various aspects thereof) in an amount which is effective to reduce cellulite.

The present invention also provides a method of caring for sensitive skin. The method comprises applying to at least parts of the skin the preparation of the present invention as set forth above (including the various aspects thereof) in an amount which is effective to care for sensitive skin.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description making apparent to those skilled in the art how the several forms of the present invention may be embodied in practice. Unless stated otherwise, all amounts, fractions and percentages given are based on the weight and the total amount or on the total weight of the preparations.

The use of preparations according to the present invention with a cosmetically effective content of anise fruit extract and white tea extract—e.g., in the form of the cited examples—surprisingly leads to a considerable improvement in the appearance of the skin. In particular, through the application of preparations according to the invention one or more of the following may be accomplished:

The barrier properties of the skin are strengthened or restored

Drying out of the skin is better counteracted

The skin is better protected against environmental influences.

Through the use of preparations according to the present invention furthermore the moisture of the skin and the deep moisture of the skin may be increased, wherein this skin moisturizing care effect and the protection from loss of moisture may last for several hours or even for the whole day or even longer.

Therefore the use of cosmetic preparations that contain a cosmetically effective amount of anise fruit extract and white tea extract for increasing the moisture of the skin or for moisturizing the skin is also according to the invention.

The preparations according to the invention furthermore are extremely suitable for the care of sensitive skin.

It was furthermore surprising that the elasticity of the skin, the hair and/or the nails and therefore cellulite or the appearance of the so-called “orange-peel skin” may be improved through the application of preparations according to the present invention.

Preferably the cosmetic or dermatological preparations according to the present invention comprise (a) at least about 0.001% by weight, e.g., at least about 0.01%, at least about 0.1%, at least about 0.5%, or at least about 1% by weight, but usually not more than about 30% by weight, e.g., not more than about 25%, not more than about 20°, not more than about 15%, not more than about 10%, or not more than about 5% by weight of anise fruit extract and (b) at least about 0.001% by weight, e.g., at least about 0.01%, at least about 0.1%, at least about 0.5%, or at least about 1% by weight, but usually not more than about 30% by weight, e.g., not more than about 25%, not more than about 20%, not more than about 15%, not more than about 10%, or not more than about 5% by weight of white tea extract, in each case based on the total weight of the preparation.

Advantageously, the weight ratio of anise fruit extract and white tea extract is from about 1:10 to about 10:1, preferably from about 1:5 to about 5:1, particularly preferably from about 1:2 to about 2:1.

It is particularly advantageous according to the present invention to employ an aqueous fruit extract from anise (INCI: Pimpinella anis fruit extract) which is rich in inorganic minerals such as sodium and magnesium ions, particularly potassium ions, and is preserved with about 0.36% of butylene glycol and about 0.14% of parabens and is obtainable through enzymatic hydrolysis of anise fruits which are solubilized in water, wherein the ratio of raw material to extract is about 1:2 and is available under the trade name BIOXYLIFT® from SILAB, (Brive Cedex, France).

Anise fruit extract (BIOXYLIFT®, SILAB) is an aqueous fruit extract from anise. The extract has an amber color and has a characteristic anise scent. It is rich in inorganic minerals such as sodium and magnesium ions, but in particular in potassium ions. The specific composition is

| Dry matter: | 40-60 g/l |
|---|---|
| Mineral ash | 11-18 g/l |
| Total protein | 12-20 g/l |
| pH | 4.5-5.5 |

The extract can be produced and stored unpreserved or preserved. Its production is described in Iinternational Patent Application WO 02/102347, the entire disclosure whereof is incorporated by reference herein.

A possible preservation is, for example, with butylene glycol 0.36% and parabens about 0.14% and is soluble in ethanol up to a ratio of 40/60 ethanol/water (v/v). In production, the anise fruits are solubilized in water and enzymatically hydrolyzed. The ratio of raw material to extract is about 1:2. Soluble and insoluble phases may be separated and subsequently filtered or subsequently subjected to sterile filtration.

White tea extract usually is obtained from tea that is fermented only to about 2%. This fermentation occurs during the wilting process in a natural manner. In the case of white tea, the delicate flower buds of this species are dried. During drying they become whitish—hence the name of this tea. It is often referred to as a special type of green tea.

White tea is now produced in almost all tea regions; apart from China, for example, also in Assam, Darjeeling, Nilgiri, Sri Lanka, Malawi, Kenya. The character differs greatly regionally and the extracts therefore also have a changing composition.

White tea belongs to the species with the INCI designation camellia sinensis. Essential constituents of white tea extract are xanthines and catechols, especially gallocatechol, epigallocatechol, epicatechol, epigallocatechol gallate, gallocatechol gallate and epicatechol gallate.

White tea extract is available, for example, from Cognis under the trade name EXTRAPONE® White Tea.

The list of the cited extraction methods of course is not intended to be limiting. Anise extract and white tea extract according to the invention can be obtained in numerous ways known per se. New ways to produce the extracts are also conceivable in principle. It is essential thereby that the anise extracts and white tea extracts show the properties according to the invention.

Typically, anise extract and white tea extract according to the present invention contain some or all of the following ingredients: flavonoids (glucosides of quercitin, campherol) sterols, tannins, amino acids, etheric oils and catechols.

The cosmetic formulations according to the present invention can be composed as usual and used for treatment of the skin and/or the hair in terms of care cosmetics. However, they can also be used in make-up products of decorative cosmetics.

Particularly preferably the preparations according to the present invention represent O/W emulsions.

Particularly advantageous preparations are furthermore obtained when antioxidants are used as additives or active ingredients. According to the invention, the preparations advantageously contain one or more antioxidants. All antioxidants suitable or usual for cosmetic and/or dermatological applications can be used as antioxidants that are favorable but nevertheless may be used optionally.

The amount of antioxidants (one or more compounds) in the preparations of the present invention is preferably from about 0.001% to about 30% by weight, particularly preferably from about 0.05% to about 20% by weight, in particular from about 1% to about 10% by weight, in each case based on the total weight of the preparation.

Preferably, the compositions of the present invention do not contain ingredients which are not conventional and/or recommended for use in cosmetic preparations. Also, the preparations of the present invention are preferably adapted for topical application to skin and as such are preferably substantially free of components which are typically used only for preparations for non-topical administration such as oral administration, parenteral administration, etc. For example, the preparations are preferably substantially free of sweetening and/or flavoring agents (e.g., sugar, artificial sweetener, syrup, natural or artificial flavors), and isotonicity imparting components. On the other hand, preparations of the present invention will often comprise one or more substances selected from perfumes, polyols such as glycerol and/or alkylene glycols, silicones, etc., i.e., substances which will not be comprised in oral, parenteral, etc. preparations.

The following examples are intended to illustrate the present invention without restricting it. The numerical values in the examples mean percentages by weight, based on the total weight of the respective preparations.

W/O Emulsions

| | Example No. | | | | |
|---|---|---|---|---|---|
| | 1 | 2 | 3 | 4 | 5 |
| Triglycerin diisostearate | 1.0 | 0.5 | 0.25 | 2.0 | 3.0 |
| Diglycerin dipolyhydroxystearate | 1.0 | 1.5 | 1.75 | 3.0 | 2.0 |
| Paraffin oil | 12.5 | 10.0 | 8.0 | 5.0 | 17.5 |
| Petrolatum | 8.0 | 6.0 | 5.0 | 12.0 | 2.5 |
| Hydrogenated Cocoglycerides | 2.0 | 1.0 | 2.5 | 5.0 | 0.25 |
| Decyl oleate | 0.5 | 0.75 | 1.0 | 2.0 | 0.25 |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Iminodisuccinic acid | 0.5 | — | — | — | 0.1 |
| Magnesium sulfate | 0.5 | 0.6 | 0.5 | 0.7 | 1.0 |
| Glycerol | 3.0 | 5.0 | 10.0 | 15.0 | 1.5 |
| White tea extract | 5 | 15 | 8 | 10 | 3 |
| Perfume | q.s. | q.s. | q.s. | q.s. | q.s. |
| Ethanol | 2.0 | — | 5.0 | — | — |
| Caprylic/Capric Triglyceride | 2.0 | 2.5 | 3.0 | 5.0 | 0.5 |
| Methyl paraben | 0.4 | 0.15 | 0.05 | 0.3 | 0.4 |
| Propyl paraben | 0.3 | 0.4 | 0.25 | 0.15 | — |
| Anise extract | 5 | 15 | 8 | 10 | 3 |
| Water | ad 100 | ad 100 | ad 100 | ad 100 | ad 100 |

| | Example No. | | | | |
|---|---|---|---|---|---|
| | 6 | 7 | 8 | 9 | 10 |
| Cetyldimethicone copolyol | 1.0 | — | — | — | 5.0 |
| PEG-30 dipolyhydroxystearate | — | 0.5 | 5.0 | — | — |
| Lanolin alcohol | 1.0 | 1.5 | 1.75 | 3.0 | — |
| Cyclomethicone + dimethicone copolyol | 10.0 | 12.5 | — | — | — |
| Cyclomethicone | 12.5 | 15 | 8.0 | — | 17.5 |
| Dimethicone | 5.0 | 13.0 | 5.0 | — | 15.0 |
| Medicinal white oil | 0.5 | 0.75 | 1.0 | 18.0 | 0.25 |
| Octyldodecanol | 0.5 | 1.0 | 0.75 | 3.0 | 0.25 |
| Panthenol | 0.5 | 1.0 | 0.75 | 0.25 | 0.1 |
| Magnesium chloride | 2.0 | 0.6 | 2.5 | 0.7 | 1.0 |
| Glycerol | 12.5 | 5.0 | 10.0 | 5.0 | 7.5 |
| White tea extract | 6 | 10 | 12 | 1.5 | 5.0 |
| Perfume | q.s. | q.s. | q.s. | q.s. | q.s. |
| Methyl paraben | 0.4 | 0.1 | 0.05 | 0.3 | 0.4 |
| Butylene glycol | — | 5.0 | — | — | 7.5 |
| Propyl paraben | 0.3 | 0.4 | 0.25 | 0.15 | — |
| Cetyldimethicone | 0.5 | — | 0.7 | — | — |
| Anise extract | 6 | 10 | 12 | 1.5 | 5.0 |
| Water | ad 100 | ad 100 | ad 100 | ad 100 | ad 100 |

O/W Emulsions

| | Example No. | | | | |
|---|---|---|---|---|---|
| | 11 | 12 | 13 | 14 | 15 |
| Glyceryl stearate citrate | 5 | — | — | — | — |
| Glyceryl stearate | — | 5 | — | — | — |
| PEG 40 stearate | — | 2 | — | — | — |
| Polyethylene glycol(21) stearyl ether | — | — | 2 | — | — |
| Polyethylene glycol(2) stearyl ether | — | — | 1 | — | — |
| Cetearyl glucoside | — | — | — | 2 | — |
| Stearic acid | — | — | — | — | 5 |
| Behenyl alcohol | — | — | — | — | 2 |
| Stearyl alcohol | 2 | 1 | — | 5 | — |
| Cetylstearyl alcohol | — | — | 2 | — | 1 |
| Hydrogenated coconut fatty glycerides | 2 | — | — | 3 | 1 |
| Butylene Glycol Caprylate/Caprate | 1 | — | 8 | — | 2 |
| Caprylic/Capric Triglycerides | — | 4 | 2 | — | — |
| Ethylhexyl coco fatty acid esters | 3 | 6 | — | — | 2 |
| Octyldodecanol | — | — | 1 | 9 | — |
| Mineral oil | 9 | 1 | 1 | 1 | 3 |
| Petrolatum | 4 | 2 | 5 | 0.5 | 0.75 |
| Glycerol | 7.5 | 15 | 65 | 25 | — |
| White tea extract | 1 | 3 | 8.5 | 0.5 | 1.5 |
| Anise extract | 1 | 3 | 8.5 | 0.5 | 2 |
| Octamethyl trisiloxane | — | 1 | 2 | 5 | — |
| Dimethylpolysiloxane | 0.5 | 0.75 | 1.25 | — | 1 |
| Dicaprylyl carbonate | 6 | 2 | 10 | 0.5 | 4 |
| Methyl paraben | 0.3 | — | 0.1 | — | 0.05 |
| Perfume | q.s. | q.s. | q.s. | q.s. | q.s. |
| Water | ad 100 | ad 100 | ad 100 | ad 100 | ad 100 |

Hydrodispersion Gels

| | Example No. | | | | |
|---|---|---|---|---|---|
| | 16 | 17 | 18 | 19 | 20 |
| Silicone oil, cyclic | 8 | 10 | 5 | 3 | — |
| Silicone oil, linear | — | — | — | — | 3 |
| Dimethicone oil | 1 | 2 | 3 | — | 3 |
| Ethanol | 10 | 15 | 7.5 | 5 | 3 |
| White tea extract | 15 | 5 | 15 | 20 | 3.0 |
| Anise extract | 15 | 5 | 15 | 20 | 3.0 |
| Sodium polyacrylate | 0.2 | 0.3 | 0.3 | 0.4 | 0.10 |
| Methyl propane diol | 2 | 3 | 4 | 5 | — |
| Glycerol | 9 | 15 | 5 | 7.5 | 25 |
| Carbomer | 0.2 | 0.3 | 0.2 | 0.4 | 0.15 |
| Acrylate/ | 0.2 | 0.15 | 0.3 | 0.4 | 0.10 |
| C10-30-alkyl acrylate crosspolymer | | | | | |
| Carrageenan (chondrus crispus) | 0.2 | — | — | 1.0 | 2 |
| Hamamelis extract | 0.1 | 0.2 | 0.3 | 0.4 | — |
| Perfume | q.s. | q.s. | q.s. | q.s. | q.s. |
| Dye | q.s. | q.s. | q.s. | q.s. | q.s. |
| Water | ad 100 | ad 100 | ad 100 | ad 100 | ad 100 |
| Aloe vera extract | 0.1 | 0.2 | 0.5 | 0.2 | 1.0 |

Ethanol Sprays

| | Example No. | | | | |
|---|---|---|---|---|---|
| | 21 | 22 | 23 | 24 | 25 |
| Glycerol | 9 | 15 | 20 | 5 | 7.5 |
| Ethanol | 42.5 | 45 | 40 | 35 | 25 |
| White tee extract | 1.5 | 10 | 5 | 2.0 | 25 |
| Anise extract | 1.5 | 10 | 5 | 2.0 | 25 |
| Water | ad 100 | ad 100 | ad 100 | ad 100 | ad 100 |
| PPG-1-PEG-9 lauryl glycol ether | 0.4 | 0.2 | 0.6 | 1.0 | 5 |
| Perfume | q.s. | q.s. | q.s. | q.s. | q.s. |
| Hamamelis extract | 0.1 | 0.2 | 0.35 | 0.4 | — |
| Aloe vera extract | 0.025 | 0.2 | 0.5 | 0.02 | 1.0 |

Skin Care Oils

| | Example No. | | | | |
|---|---|---|---|---|---|
| | 26 | 27 | 28 | 29 | 30 |
| Jojoba oil | 5.0 | 7.5 | 10.0 | 25 | 25 |
| Hydrogenated PEG 40 castor oil | 2.5 | 7.5 | 10.0 | 1.25 | 5 |
| Macademia nut oil | 5.0 | 10.0 | 20.0 | 25.0 | 5.0 |
| Anise extract | 1.0 | 2.5 | 2.0 | 0.1 | 0.25 |
| Cyclomethicone | — | — | — | — | 1.5 |
| Dimethicone | — | 0.25 | — | — | — |
| White tea extract | 1.0 | 2.5 | 0.75 | 0.25 | 1.5 |
| Medicinal white oil | 30.0 | — | — | — | 5.0 |
| Caprylic/capric triglyceride | 5.0 | — | — | — | — |
| Castor oil | 5.0 | — | — | — | 5.0 |
| Octyldodecanol | — | 1.0 | — | 10.5 | 0.25 |
| Perfume | q.s. | q.s. | q.s. | q.s. | q.s. |
| Isopropyl palmitate | — | — | — | — | — |
| Ethylhexyl stearate | 10.0 | — | — | — | — |
| Tocopherol acetate | 0.1 | — | — | 0.15 | — |
| Tocopherol | 0.05 | 0.15 | 0.05 | 0.05 | 0.05 |
| Sunflower oil | ad 100 | ad 100 | ad 100 | ad 100 | ad 100 |

O/W Emulsions, light

| | Example No. | | | | |
|---|---|---|---|---|---|
| | 31 | 32 | 33 | 34 | 35 |
| White tea extract | 15 | 10 | 5 | 20 | 30 |
| Glyceryl stearate | 1 | 1 | 2 | 5 | 8 |
| Cetearyl alcohol + sodium cetearyl sulfate | 3 | 4 | 5 | 2 | 8 |
| Anise extract | 15 | 10 | 5 | 20 | 30 |
| Xanthan gum | 0.5 | 0.75 | 0.35 | 0.4 | 1.0 |
| Perfume | q.s. | q.s. | q.s. | q.s. | q.s. |
| Hamamelis extract | 0.1 | 0.2 | 0.35 | 0.4 | — |
| Aloe vera extract | 0.025 | 0.2 | 0.5 | 0.02 | 1.0 |
| Dicaprylyl carbonate | 5 | 7.5 | 3 | 10 | 2.5 |
| Caprylic/capric triglyceride | 5 | 7.5 | 3 | 2.1 | 1.8 |
| Silicone oil, linear | 3 | 2 | — | 0.75 | 5 |
| Caprylyl glycol | 0.5 | — | 0.3 | — | 0.1 |
| Water | ad 100 | ad 100 | ad 100 | ad 100 | ad 100 |
| Ethanol | 2.5 | 3.5 | 5 | 1.5 | — |

Gels

| | Example No. | | | | |
|---|---|---|---|---|---|
| | 36 | 37 | 38 | 39 | 40 |
| Polyacrylic acid, Na salt | 1.0 | 0.2 | 0.25 | 0.1 | 1.5 |
| PEG-40 stearate | 0.5 | — | — | 0.75 | — |
| Carrageenan | 1.25 | 0.2 | 0.2 | 0.1 | 2.5 |
| Sodium polyacrylate | 0.2 | 0.2 | 0.2 | 0.5 | 1.5 |
| Vinyl pyrrolidone/vinyl acetate copolymers | 1.0 | — | — | 0.2 | — |
| Acrylate/C10-30-alkyl acrylate | 1.5 | 0.2 | 0.25 | 1.5 | 0.75 |
| crosspolymer | | | | | |
| Water-soluble Blue (Cl 42090) | 0.001 | 0.4 | 0.0001 | 0.003 | — |
| Water-soluble Yellow (Cl 10316) | 0.01 | 0.004 | 0.0001 | — | 0.002 |
| Cyclomethicone | 1 | 8.5 | 8.0 | 0.5 | 3 |
| Dimethiconol | 0.5 | 0.5 | 1.0 | 1.25 | 1.0 |
| Cetylstearyl alcohol | — | — | — | 1 | — |
| Hydrogenated polyisobutene | 0.5 | — | — | — | 0.25 |
| White tea extract | 0.5 | 0.1 | 0.75 | 0.5 | 0.25 |
| Anise extract | 1.0 | 2.0 | 0.5 | | |
| Panthenol | 0.5 | — | — | 0.25 | 0.1 |
| Glycerol | 3.0 | 8.6 | 10.0 | 17.2 | 5.0 |
| Perfume | q.s. | q.s. | q.s. | q.s. | q.s. |
| Methyl propanediol | 4.5 | 2.0 | 2.0 | 1.5 | — |
| Phenoxyethanol | 0.1 | 1.0 | 0.3 | — | 0.5 |
| Propyl paraben | 0.3 | — | — | 0.15 | — |
| Ethanol | — | 10.0 | 10.0 | — | 3.5 |
| Water | ad 100 | ad 100 | ad 100 | ad 100 | ad 100 |

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. An active substance combination, wherein the combination comprises anise fruit extract and white tea extract.

2. A cosmetic preparation, wherein the preparation comprises an effective amount of the active substance combination of claim 1.

3. The preparation of claim 2, wherein the preparation comprises from about 0.0001% to about 30% by weight of anise fruit extract, based on a total weight of the preparation.

4. The preparation of claim 3, wherein the preparation comprises from about 0.5% to about 10% by weight of anise fruit extract.

5. The preparation of claim 2, wherein the preparation comprises from about 0.0001% to about 30% by weight of white tea extract, based on a total weight of the preparation.

6. The preparation of claim 5, wherein the preparation comprises from about 0.5% to about 10% by weight of white tea extract.

7. The preparation of claim 2, wherein a weight ratio anise fruit extract:white tea extract is from about 1:10 to about 10:1.

8. The preparation of claim 7, wherein the ratio is from about 1:5 to about 5:1.

9. The preparation of claim 7, wherein the ratio is from about 1:2 to about 2:1.

10. The preparation of claim 2, wherein the preparation comprises anise fruit extract and one or more substances selected from gallocatechol, epigallocatechol, epicatechol, epigallocatechol gallate, gallocatechol gallate, epicatechol gallate, catechol, quercetin, and methyl xanthine.

11. The preparation of claim 10, wherein the methyl xanthine comprises one or more of caffeine, theophylline and theobromine.

12. The preparation of claim 2, wherein the preparation comprises an aqueous anise fruit extract.

13. The preparation of claim 2, wherein the preparation is present as an emulsion.

14. A cosmetic preparation, wherein the preparation comprises from about 0.1% to about 30% by weight of an aqueous anise fruit extract and from about 0.1% to about 30% by weight of white tea extract, based on a total weight of the preparation, a weight ratio anise fruit extract:weight tea extract being from about 5:1 to about 1:5.

15. The preparation of claim 14, wherein the preparation comprises from about 0.5% to about 20% by weight of an aqueous anise fruit extract and from about 0.5% to about 20% by weight of white tea extract.

16. The preparation of claim 15, wherein the preparation is present as an O/W emulsion.

17. A method of increasing the moisture content of skin or moisturizing skin, wherein the method comprises applying to at least parts of the skin the preparation of claim 2 in an amount which is effective to at least one of increase the moisture content of the skin and moisturize the skin.

18. A method of improving the surface structure of skin, wherein the method comprises applying to at least parts of the skin the preparation of claim 2 in an amount which is effective to improve the surface structure of the skin.

19. A method of reducing cellulite, wherein the method comprises applying to at least parts of the skin the preparation of claim 2 in an amount which is effective to reduce cellulite.

20. A method of caring for sensitive skin, wherein the method comprises applying to at least parts of the skin the preparation of claim 2 in an amount which is effective to care for sensitive skin.
